(19) United States
(12) Patent Application Publication
(10) Pub. No.: US 2013/0289429 A1
(43) Pub. Date: Oct. 31, 2013

(54) ELECTROCARDIOGRAPH

(71) Applicant: GENERAL ELECTRIC COMPANY, Scenectady, NY (US)

(72) Inventors: Ryan Jerome RAMOS, Waukesha, WI (US); Huei Mian CHIAH, ShangHai (CN); Duncan Lovel TREVOR-WILSON, ShangHai (CN); Jun HOU, ShangHai (CN)

(73) Assignee: General Electric Company, Scenectady, NY (US)

(21) Appl. No.: 13/867,404

(22) Filed: Apr. 22, 2013

(30) Foreign Application Priority Data
Apr. 27, 2012 (CN) 201210143392.8

(51) Int. Cl. A61B 5/0402

(52) U.S. Cl. CPC A61B 5/0402
USPC 600/523

(57) ABSTRACT

An electrocardiograph comprising a base part and an operating and displaying part. The operating and displaying part is disposed on the base part and includes a displaying unit and an operating unit. The displaying unit is connected to the operating unit. The operating and displaying part is coupled with the base part via a rotating structure such that the operating and displaying part can rotate left and right relative to the base part. Since the operating and displaying part as described in the present application can rotate left and right relative to the base part, the user can adjust the view angle of the operating and displaying part as required.

### BACKGROUND OF THE INVENTION

The present application relates to the field of electrocardiography system for making monitoring and diagnosis on human body, and more particularly, to an electrocardiograph.

Electrocardiograph is an instrument for discovering and diagnosing irregular phenomena of heart, which measures a surface potential on human body and records a current associated with heart muscle activity; furthermore, it may provide electrocardiographic data about the measured human body.

FIG. 1 is a prior art electrocardiograph published in U.S. Pat. No. 4,974,599. The electrocardiograph 12 comprises a substantially rectangular main body 14, the upper surface of which is provided with a display assembly 16, a power source control switch 18, and a plurality of other switches 110. The display assembly 16 is mounted in the main body 14, and all of the switches 18 and 110 line up beneath the display assembly 16.

Such electrocardiograph 12 has the following shortcomings. Firstly, since the display assembly 16 is mounted in the main body 14, it is not so convenient for a user. For example, the user may need to adjust the angle of the electrocardiograph 12 to view the content displayed by the display assembly 16. Secondly, the setting of switches of the electrocardiograph 12 is relatively simple, and accordingly the user may hardly handle the operation procedure well.

FIG. 2 is an electrocardiograph system published in U.S. Pat. No. 7,236,818. The electrocardiograph system 210 comprises an acquisition unit 212 and a processing and displaying unit 214. The processing and displaying unit 214 may be equivalent to the above mentioned electrocardiograph 12, which is configured to be hand-held and has a display 220 fixed thereon. Since the processing and displaying unit 214 is configured to be hand-held, it is convenient for an operator to view the display 220.

Additionally, a product MAC 1200 ST purchased from General Electric Company may also be referred to as the relevant electrocardiograph, which is also portable.

Though the above relevant electrocardiographs are configured to be hand-held to facilitate the operator's view of the display, continuous improvement of electrocardiograph is still required.

### BRIEF DESCRIPTION OF THE INVENTION

In order to overcome the disadvantages of the electrocardiograph of prior art, the present application provides an electrocardiograph.

An embodiment of the present invention is directed to an electrocardiograph comprising a base part and an operating and displaying part is disposed on the base part. The operating and displaying unit includes an operating unit and a displaying unit connected to the operating unit. The operating and displaying part is coupled with the base part via a rotating structure such that the operating and displaying part can rotate left and right relative to the base part.

In an embodiment of the present invention, the base part comprises an upper housing and a lower housing which form a space.

In an embodiment of the present invention, the base part comprises a circuit device configured to generate a diagnosis electrocardiogram, and the circuit device is received within the space.

In an embodiment of the present invention, the rotating structure comprises a passage therein for receiving a wire connecting the base part with the operating and displaying part.

In an embodiment of the present invention, the rotating structure comprises at least one elastic locking arm. The locking arm protrudes from one of the base part and the operating and displaying part and matches with an opening of the other of the base part and the operating and displaying part, such that the operating and displaying part can rotate left and right relative to the base part, but movement of the operating and displaying part away from the base part is restricted.

In an embodiment of the present invention, the displaying unit is integrally formed with the operating unit, and the displaying unit forms an angle greater than about 90° and less than about 180° relative to the operating unit.

In an embodiment of the present invention, the operating and displaying part rotates left and right in a range between about 0° and about 360° relative to the base part.

In an embodiment of the present invention, the electrocardiograph further comprises a stopping structure for restricting the left and right rotation of the operating and displaying part to a range only between about 0° and about 360° relative to the base part.

In an embodiment of the present invention, the operating unit comprises a function key region and a number and letter key region.

In an embodiment of the present invention, the displaying unit is connected to the operating unit via a hinge, such that the displaying unit can rotate up and down relative to the operating unit.

Comparing to the prior art, the operating and displaying part as described in an embodiment of the present invention, can rotate left and right relative to the base part so that a user/operator can adjust the view angle of the operating and displaying part as required. Further, since the operating unit is provided with a function key region and a number and letter key region, the user can handle the operation procedure of the electrocardiograph without any difficulty.

Various other features and advantages of the invention will be made apparent to those skilled in the art from the accompanying drawings and detailed description thereof.

### BRIEF DESCRIPTION OF THE DRAWING

Embodiments of the present invention will be described in more detail by referring to attached drawings which are not necessarily drawn to scale, wherein:

FIG. 1 is a schematic view illustrating an electrocardiograph of prior art;

FIG. 2 is a schematic view illustrating use condition of an electrocardiograph system of prior art;

FIG. 3 is a schematic three dimensional view illustrating the electrocardiograph according to an embodiment of the present invention, which shows that the electrocardiograph is in a state of use;

FIG. 4 is a schematic three dimensional view illustrating another state of use of the electrocardiograph shown in FIG. 3, which shows that the operating and displaying part of the electrocardiograph has been rotated 90° as compared with the position as shown in FIG. 3;

FIG. 5 is a schematic view illustrating the section of a rotating structure of the electrocardiograph according to an embodiment of the present invention;

FIG. 6 is a schematic view illustrating the section of a stopping structure of the electrocardiograph according to an embodiment of the present invention; and

FIG. 7 is a schematic plan view illustrating a keyboard design of an operating unit of the electrocardiograph according to an embodiment of the present invention.

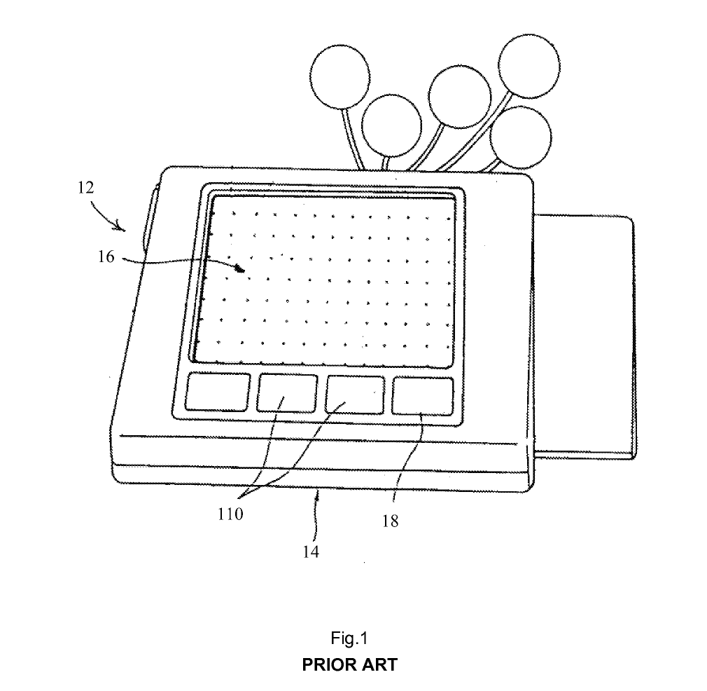

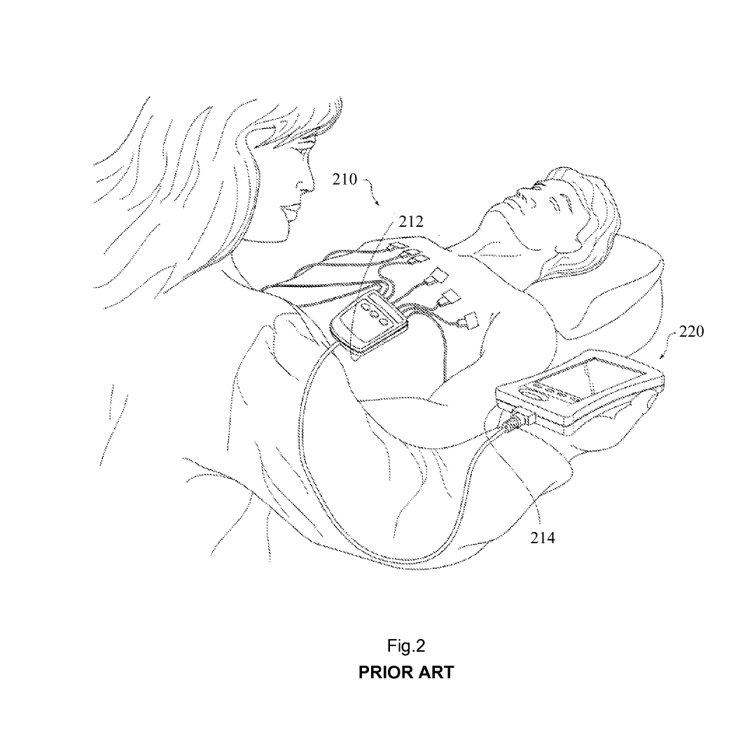

### DETAILED DESCRIPTION OF THE INVENTION

Embodiments of the present invention will be described in more detail in conjunction with embodiments. Those skilled in the art should understand that these embodiments are examples, and are not intended to limit the present invention and its scope.

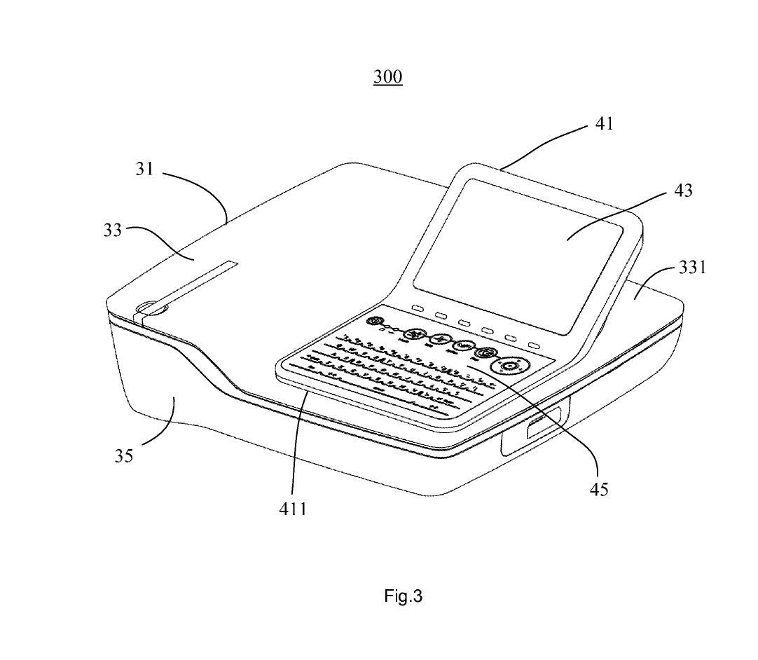

Referring to FIG. 3, an electrocardiograph 300 according to an embodiment of the present invention is shown. The electrocardiograph 300 comprises a base part 31, and an operating and displaying part 41. The operating and displaying part 41 is disposed on the base part 31.

The base part 31 may comprise an upper housing 33 and a lower housing 35, the both of which form a receiving space. The base part 31 usually comprises a circuit device (not shown) configured to generate diagnosis electrocardiogram, the circuit device being received within the receiving space. The upper housing 33 has an upper surface 331 that may be designed to be a planar or streamlined surface as required.

The operating and displaying part 41 comprises a displaying unit 43 and an operating unit 45. The displaying unit 43 may be connected to the operating unit 45. The operating and displaying part 41 has a lower surface 411. The operating and displaying part 41 is disposed on the base part 31 by attaching the lower surface 411 to a portion of the upper surface 331. According to an embodiment of the present invention, the displaying unit 43 may be integrally formed with the operating unit 45, and furthermore, it forms an angle greater than 90° and less than 180° relative to the operating unit. Alternatively, the displaying unit 43 may be connected to the operating unit 45 via a hinge, such that the displaying unit 43 can rotate up and down relative to the operating unit 45 (that is, the inclination between the displaying unit 43 and the operating unit 45 is adjustable).

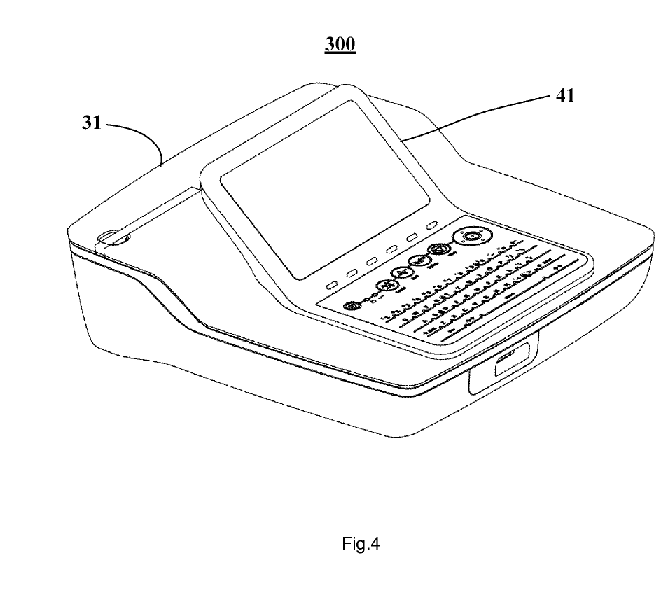

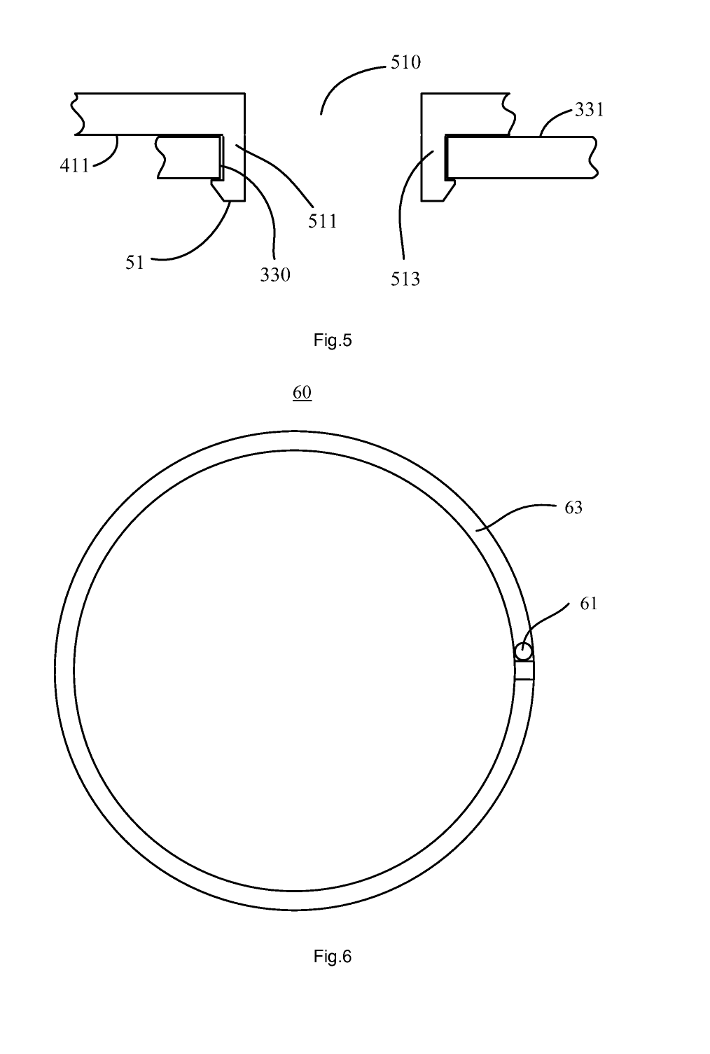

Referring to FIG. 4, it shows that the operating and displaying part 41 of the electrocardiograph 300 can rotate left and right relative to the base part 31. The left and right rotation can be implemented through the rotating structure as shown in FIG. 5.

As shown in FIGS. 4 and 5, the operating and displaying part 41 is coupled with the base part 31 via a rotating structure (e.g., a pivot 51) such that the operating and displaying part 41 can rotate left and right relative to the base part 31. The pivot 51 may comprise a passage 510 therein for receiving a wire and/or communication wire (not shown) connecting the base part 31 with the operating and displaying part 41. The pivot 51 includes two elastic locking arms 511 and 513, which protrude from the lower surface 411 of the operating and displaying part 41 and match with an opening 330 of the upper housing 33 of the base part 31, such that the operating and displaying part 41 can rotate left and right relative to the base part 31 and movement of the operating and displaying part 41 away from the base part 31 is restricted. Of course, the number of elastic locking arms may be one or more than three, and the positions of the elastic locking arms 511 and 513 and the opening 330 are interchangeable (that is, the elastic locking arms 511 and 513 can be disposed on the upper housing 33, and the opening 330 can be disposed on the operating and displaying part 41).

As shown in FIGS. 3 and 6, the operating and displaying part 41 can restrict, via a stopping structure 60, the left and right rotation thereof to a range only between 0° and 360° relative to the base part. Specifically speaking, the stopping structure 60 may include a bump 61 protruding from the lower surface 411 and an annular groove 63 arranged on the upper surface 331. The bump 61 can rotate within the annular groove 63 by an angle of less than 360°. Of course, the positions of the bump 61 and the annular groove 63 are interchangeable, and furthermore, the rotation angle can vary with the radian of the annular groove 63 (for example, setting the rotation angle to be 90°).

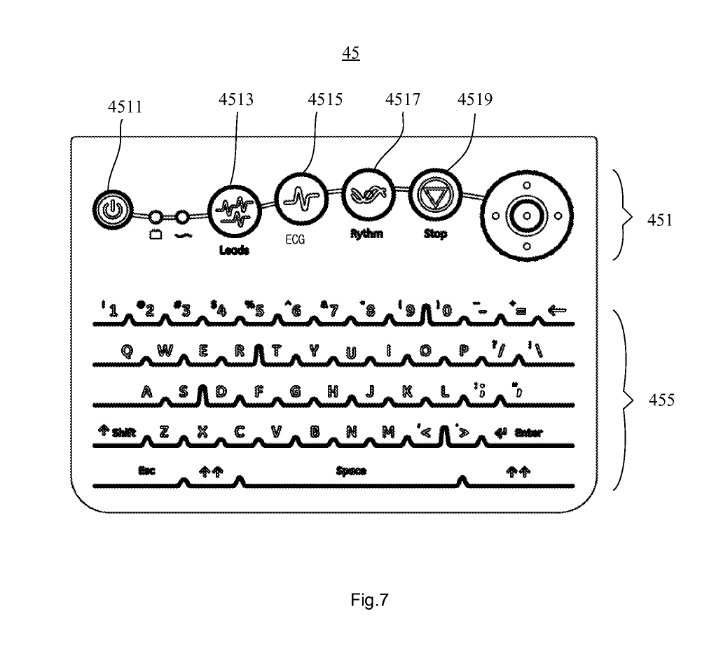

As shown in FIG. 7, it is a schematic plan view illustrating a keyboard design of an operating unit of the electrocardiograph according to an embodiment of the present invention. The operating unit 45 can comprise a function key region 451 and a number and letter key region 455 to facilitate user's operation. The function key region 451 may include in sequence a power key 4511, a leads connecting key 4513, an electrocardiogram (ECG) measuring key 4515, a rhythm measuring key 4517, a stop key 4519, etc. Of course, it is also allowed to arrange a subfunction key region between the function key region 451 and the number and letter key region 455 to further subdivide the operations.

The aforementioned embodiments of the present invention relate to an electrocardiograph for addressing the problem that the electrocardiograph of prior art is inconvenient. Compared to the prior art, the operating and displaying part as described herein can rotate left and right relative to the base part so that the user can adjust the view angle of the operating and displaying part as required. Further, since the operating unit is provided with a function key region and a number and letter key region, the user can handle the operation procedure of the electrocardiograph without any difficulty.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An electrocardiograph, comprising:
a base part; and
an operating and displaying part disposed on the base part, the operating and displaying part comprising an operating unit and a displaying unit connected to the operating unit;
wherein the operating and displaying part is coupled with the base part by a rotating structure and wherein the operating and displaying part can rotate left and right relative to the base part.

2. The electrocardiograph according to claim 1, wherein the base part comprises an upper housing and a lower housing, wherein the upper housing and lower housing form a space.

3. The electrocardiograph according to claim 2, wherein the base part comprises a circuit device configured to generate a diagnosis electrocardiogram, wherein the circuit device is received within the space.

4. The electrocardiograph according to claim 1, wherein the rotating structure comprises a passage configured to receive a wire connecting the base part with the operating and displaying part.

5. The electrocardiograph according to claim 1, wherein the rotating structure comprises at least one elastic locking arm that protrudes from one of the base part and the operating and displaying part and matches with an opening of the other of the base part and the operating and displaying part, such that the operating and displaying part can rotate left and right relative to the base part but is restricted from moving away from the base part.

6. The electrocardiograph according to claim 1, wherein the displaying unit is integrally formed with the operating unit, and the displaying unit forms an angle greater than 90° and less than 180° relative to the operating unit.

7. The electrocardiograph according to claim 1, wherein the operating and displaying part rotates left and right in a range of between 0° and 360° relative to the base part.

8. The electrocardiograph according to claim 7, wherein the electrocardiograph further comprises a stopping structure configured to restrict the left and right rotation of the operating and displaying part to a range between 0° and 360° relative to the base part.

9. The electrocardiograph according to claim 1, wherein the operating unit comprises a function key region and a number and letter key region.

10. The electrocardiograph according to claim 1, wherein the displaying unit is connected to the operating unit by a hinge, such that the displaying unit can rotate up and down relative to the operating unit.
